(19) United States
(12) Patent Application Publication
(10) Pub. No.: US 2022/0031959 A1
(43) Pub. Date: Feb. 3, 2022

(54) PEN NEEDLE APPARATUS

(71) Applicant: Becton, Dickinson and Company, Franklin Lakes, NJ (US)

(72) Inventors: Xi ZENG, Suzhou, Jiangsu (CN); Huasheng HUANG, Suzhou, Jiangsu (CN); Wei HU, Shanghai (CN); Junyu ZHOU, Shanghai (CN)

(21) Appl. No.: 17/276,719

(22) PCT Filed: Jul. 31, 2019
(86) PCT No.: PCT/US2019/044437

§ 371 Date: Mar. 16, 2021

(30) Foreign Application Priority Data
Sep. 17, 2018 (CN) 201811078215.X
Sep. 17, 2018 (CN) 201821509867.X

(51) Int. Cl. A61M 5/32

(52) U.S. Cl. CPC A61M 5/3245; A61M 2005/3254

(57) ABSTRACT

A pen needle apparatus includes a housing (32), a movable gripping mechanism (34) for gripping a pen needle assembly (18), and an actuator (36). The gripping mechanism (34) includes first and second pivoting arms (44) that pivot between a first closed position and a second open position. In the closed position, the arms engage and grip the outer cover (22) of the pen needle assembly (18) so that the needle hub (16) can be separated from the outer cover (22) and the pen needle placed back in the outer cover (22) after use of the pen needle. The actuator (36) is connected to the arms (44) where movement of the actuator pivots the arms to a second open position to release the outer cover of the pen needle assembly. The arms include a gripping surface for engaging the inner shield (24) of the pen needle assembly to assist in removing the inner shield (24) from the needle hub (16).

### FIELD OF THE INVENTION

The present invention relates generally to an apparatus for use in mounting and attaching a pen needle assembly to a delivery device, such as a pen delivery device. The apparatus has a gripping mechanism for retaining the outer cover of a needle hub to assist in removing the outer cover and the inner shield from the needle hub, and disposing of the used needle hub after use.

### BACKGROUND OF THE INVENTION

Insulin and other injectable medications are commonly delivered with drug delivery pens, whereby a disposable pen needle hub is attached to the pen to facilitate drug container access and allow fluid egress from the container through the needle into the patient.

Various pen needle delivery devices are known in the art for dispensing the substance to the patient. The delivery devices often use a disposable needle hub having a cannula or needle extending from a patient end of the hub for inserting into the patient. A non-patient end of the hub is coupled to the pen delivery device for delivering the substance to the patient.

The needle hub assembly is often packaged in a container containing several loose needle hubs. A needle hub is selected from the package and attached to the pen needle delivery device for injecting the patient and then removed to be discarded. The needle hub package includes an outer cover that encloses the needle hub and a removable seal that is peeled from the outer cover to open the cavity so that the needle hub can be removed. The needle hub can have threaded non-patient end that is threaded onto the delivery device. The delivery device with the attached needle hub is then removed from the outer cover. An inner needle shield is attached to the needle hub to cover the cannula until the device is ready for use. The shield is removed to expose the cannula for use to deliver the substance to the patient. After use, the needle hub can be inserted back into the outer cover to enclose the exposed cannula. The pen delivery device is separated from the needle hub leaving the needle hub within the outer cover.

The prior devices require the use of both hands to connect to and remove the needle hub from the delivery device. During the placement back into the outer cover, the cannula is exposed and provides an increased risk of accidental needle stick. The manual operation of holding the outer cover while positioning the spent needle hub and cannula into the cavity of the outer cover can be difficult without accidental needle stick.

Existing pen needle assemblies are disclosed in U.S. Patent Application Publication Nos. 2006/0229562 to Marsh et at and 2007/0149924 to R. Marsh, the entire contents of both of which are hereby incorporated by reference.

Although the prior devices have been suitable for the intended use, there is a continuing need in the industry for improved packaging for a pen needle hub assembly.

### SUMMARY

The present invention is directed to a pen needle apparatus and device for receiving and supporting at least one pen needle assembly for attaching the pen needle to a delivery device, such as a delivery pen. In particular, the apparatus is used with a pen needle assembly that can reduce the risk of inadvertent needle stick while attaching and removing the pen needle from the delivery pen and disposing of the used pen needle.

The apparatus includes a housing, a movable gripping mechanism assembly, and an actuator to operate the gripping mechanism assembly. In one embodiment, the apparatus is provided with an opening and a well for a pen needle assembly. The pen needle assembly includes an outer cover, a pen needle hub supporting a cannula or needle, and an inner shield covering the cannula. A removable seal closes the open end of the outer cover where the seal is removed before attaching the needle hub to the delivery pen. The apparatus retains and supports the pen needle assembly and the needle hub and cannula while attaching to a delivery pen or other delivery device. The apparatus is able to store a used needle hub and the outer cover for disposal. The gripping mechanism of the apparatus has a recess forming a well for receiving and retaining the inner shield that covers the cannula so that the inner shield can be removed from the needle hub without the operator handling the inner shield. The used needle hub can be inserted back into the outer cover and transferred to a storage tray or internal sharps container for disposal. The inner shield can be re-attached to the needle hub or discarded in the storage tray. The actuator operates the gripping mechanism assembly and releases the needle hub, outer cover, and inner shield to a suitable disposal tray, bin or container, such as a sharps container.

In one embodiment, the inner shield and outer cover can be inserted into a respective opening and a well in the apparatus to engage a gripping mechanism within the apparatus so that the inner shield and outer cover are retained by a friction fit or interference fit. The needle hub and cannula can then be pulled away to separate the inner shield while the inner shield is retained in the well without the need for the user to handle the inner shield. The gripping mechanism can release the inner shield and outer cover to drop into a tray or cavity in the apparatus that forms a sharps container.

The apparatus or device is constructed for receiving and supporting a pen needle assembly to attach the needle hub to the delivery device without the need for the user to handle the exposed hub with the exposed cannula on the needle hub. The pen needle assembly is positioned in the apparatus so that the delivery device can be attached to the non-patient end of the needle hub. An inner needle shield on the needle hub can then be inserted into a well in the apparatus to grip the inner shield so that the inner shield can be separated from the needle hub without handling the inner shield. After use, the needle hub is positioned in the outer cover that is retained in the well of the device and disconnected from the delivery device where the device retains the used needle hub and outer cover.

The gripping mechanism includes pivoting arms that cooperate to grip the outer cover and inner shield during use of the pen needle. An actuator, such as a button, has cam surfaces that engage the arms so that linear movement of the actuator pivots the arms away from each other to an open position to release the inner shield and outer cover.

The apparatus includes a housing having an internal cavity for receiving and storing a used pen needle assembly that includes the needle hub and needle. The housing has a gripping mechanism with at least one gripping member, such as a pivoting arm, that is able to grip the outer cover of a pen needle so that the needle hub can be attached to the delivery pen. The used needle hub can be inserted back into the outer cover that is retained in the apparatus and separated from the delivery pen. An actuator is actuated to release the outer cover so that the outer cover and used needle hub can be removed from the apparatus or deposited directly into a storage tray or other storage compartment in the device for storing the used pen needle assembly until ready for disposal.

A pen needle apparatus in one aspect comprises a housing, a mechanism, and an actuator. The housing has an inner cavity, a top end, and a bottom end. The movable gripping assembly has at least one pivoting arm configured for gripping an outer cover of a pen needle assembly when the gripping assembly is in a first closed position and for releasing the outer cover when the gripping assembly is in a second open position. The actuator connects to the pivoting arms in the housing to move the pivoting arm between the first position and the second position to release the outer cover.

The gripping mechanism in one embodiment includes at least one and typically two pivoting arms that can grip and retain the outer cover and can pivot away from each other to release the outer cover. The assembly includes the actuator, such as a movable button or side portion, that includes a mechanism to pivot the arms outwardly to release the outer cover. The actuator can have a slot forming a cam surface that cooperates with a cam follower on the arms so that movement of the actuator imparts pivoting movement to the arms to enable the arms to pivot between an open position and closed position. The arms and the actuator can be spring biased in one embodiment to return the arms and actuator to the starting position.

The features are further provided by a pen needle assembly apparatus for receiving a pen needle assembly, where the apparatus includes a housing, a gripping mechanism, and an actuator. The housing has an inner cavity with a storage compartment, a top end configured for receiving a pen needle assembly, and a bottom end. The gripping mechanism is coupled to the housing and oriented below the top end. The gripping mechanism has a first arm pivotally connected to the housing and a second arm pivotally connected to the housing and configured for gripping the outer cover of the pen needle assembly when the arms are in a first position. The arms are movable to a second position to release the outer cover. The actuator is coupled to the housing for sliding in a transverse direction toward the gripping mechanism. The actuator is coupled to the first arm and the second arm for pivoting the first arm and the second arm from the first closed position to a second open position to release the outer cover from the apparatus.

After use of the needle huh and cannula, the needle huh while attached to the delivery pen is inserted into the opening and into the outer cover where the needle hub can be gripped by a friction fit or interference fit to the outer cover so that the pen needle delivery device can be separated from the needle hub without the operator handling the needle hub thereby reducing the risk of inadvertent needle stick.

The features of the apparatus are further attained by a method of coupling the needle hub of a pen needle assembly to a delivery device using a pen needle assembly apparatus. The apparatus has a top wall with an opening and a gripping mechanism supporting the pen needle assembly, and an actuator to operate the gripping mechanism. The method includes the steps of inserting the end of the delivery device into a pen needle assembly that is retained in a first well of the gripping mechanism and coupling to the open end of the needle hub. The delivery device with the needle hub attached is removed leaving the outer cover retained by the gripping mechanism. The inner shield is inserted into a well where the inner shield is gripped with sufficient force that the needle hub can be pulled free and separated from the inner shield to expose the cannula for use in delivering the substance to the patient. After use, the needle hub is inserted into the outer cover that was retained in the first well and disconnected from the delivery device. The actuator is then actuated to release the used needle hub and outer cover from the gripping mechanism.

The objects, advantages, and salient features of the invention will become apparent from the following detailed description, which, taken in conjunction with the annexed drawings, discloses exemplary embodiments of the invention.

### BRIEF DESCRIPTION OF THE DRAWINGS

The above benefits and other advantages of the various embodiments of the present invention will be more apparent from the following detailed description of exemplary embodiments of the present invention and from the accompanying figures, in which:

FIG. 1 is an exploded perspective view of a pen needle delivery device in one embodiment showing the pen needle assembly that includes a needle hub supporting a cannula, inner shield, and outer cover;

FIG. 2 is a cross-sectional view of the pen needle assembly;

FIG. 3 is a perspective view of the pen needle apparatus and storage tray in one embodiment of the invention;

FIG. 4 is an exploded view of the pen needle apparatus;

FIG. 5 is a top perspective view of the apparatus of FIG. 3 with the top wall removed showing the gripping mechanism;

FIG. 6 is a perspective view of the apparatus;

FIG. 7 is a top perspective view of the arm of the gripping mechanism;

FIG. 8 is a bottom perspective view of the arms of the gripping mechanism;

FIG. 9 is a partial cross-sectional view of the arms of the gripping mechanism;

FIG. 10 is perspective view of the spring clips for the arms;

FIG. 11 is a top perspective view of the arms supporting an outer cover of the pen needle;

FIG. 12 is a perspective view of the arms of the gripping mechanism showing the well for the inner shield;

FIG. 13 is a perspective view showing the spring clips gripping the inner shield;

FIG. 14 is a cross-sectional view of the apparatus showing the arm of the gripping mechanism;

FIG. 15 is a perspective view showing the actuator in a position with the movable arms in the open position;

FIG. 16 is a perspective view showing the pen needle inserted into the apparatus for removing the outer cover;

FIG. 17 is a perspective view showing a pen needle removed from the apparatus;

FIG. 18 is a perspective view showing the inner shield of the pen needle inserted into the apparatus;

FIG. 19 is a perspective view showing the inner shield retained in the apparatus;

FIG. 20 is a perspective view of the tray for receiving the used pen needles; and

FIG. 21 is cross sectional view of the apparatus showing the spring for biasing the actuator and gripping mechanism to the closed position.

Throughout the drawings, like reference numbers will be understood to refer to like parts, components, and structures.

### DETAILED DESCRIPTION OF THE EXEMPLARY EMBODIMENTS

The present invention is directed to an apparatus or device for use with a pen needle assembly for storing and supporting at least one pen needle assembly for use with a pen delivery device. The device stores used pen needles where the used needle hub can be received in the device after use and later discarded without handling the needle hub to reduce the risk of accidental needle stick.

Reference will now be made in detail to embodiments of the present invention, which are illustrated in the accompanying drawings, wherein like reference numerals refer to like elements throughout. The embodiments described herein exemplify, but do not limit, the present invention by referring to the drawings. The exemplary embodiments are presented in separate descriptions, although the individual features and construction of these embodiments can be combined in any number of ways to meet the therapeutic needs of the user.

It will be understood by one skilled in the art that this disclosure is not limited in its application to the details of construction and the arrangement of components set forth in the following description or illustrated in the drawings. The embodiments herein are capable of being modified, practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. The term substantially refers to largely but not necessarily entirely. Further, terms such as up, down, bottom, and top are relative, and are employed to aid illustration, but are not limiting.

The apparatus for the needle hub assembly enables the convenient and easy assembly and removal of the needle hub on the pen delivery device with reduced handling of the exposed needle cannula. The device for the pen needle assembly can be placed on a flat surface, such as a table, so that the pen needle delivery device can be attached to the needle hub with one hand thereby reducing the risk of the needle stick by handling the needle hub with the exposed needle cannula.

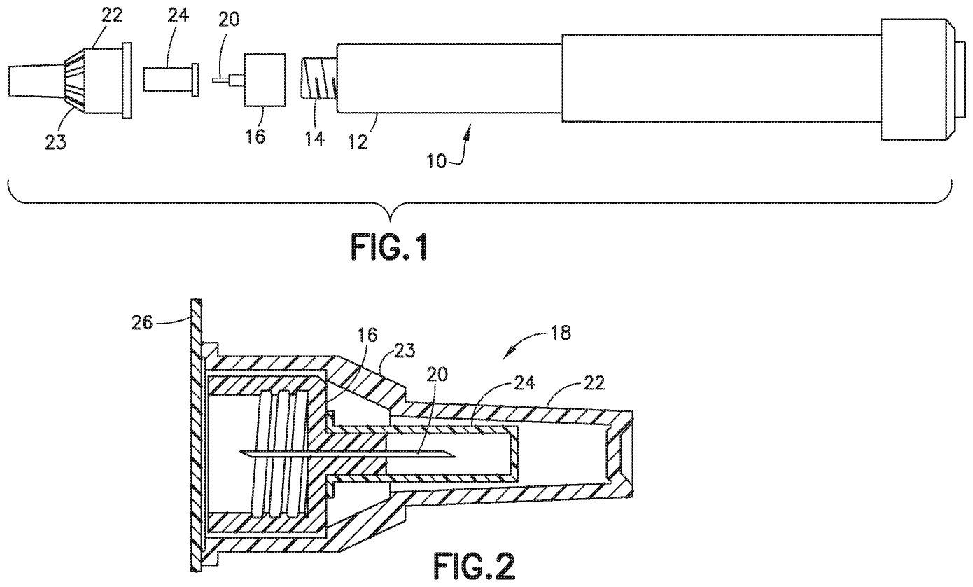

Pen needle delivery device 10, as shown in FIG. 1 typically comprises a dose knob/button, an outer sleeve 12, and a cap. A dose knob/button allows a user to set the dosage of medication to be injected. The outer sleeve 12 is gripped by the user when injecting medication. The cap is used by the user to securely hold the pen injector device 10 in a shirt pocket or other suitable location and provide cover/protection from accidental needle injury.

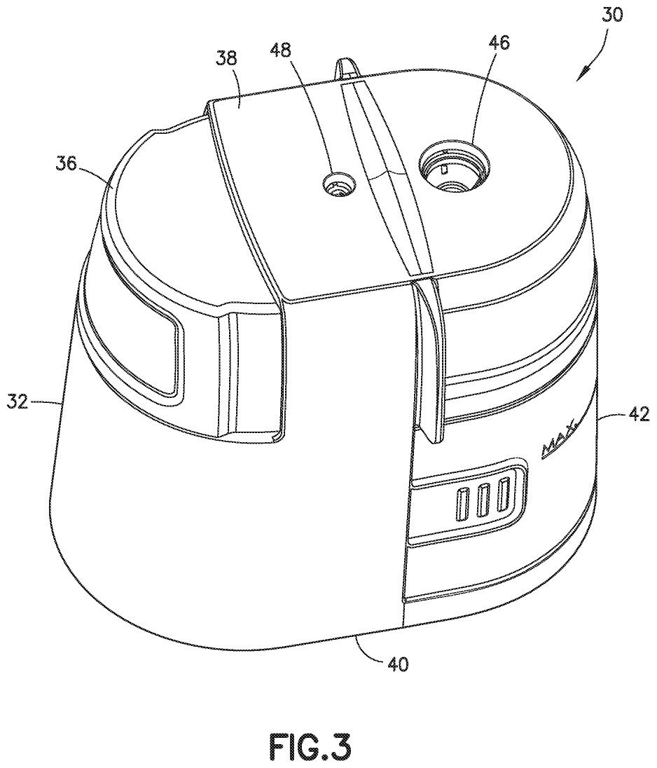

In standard pen needle delivery devices the dosing and delivery mechanisms are all found within the outer sleeve 12 and is not described in greater detail here as they are understood by those knowledgeable of the art. A medicament cartridge is typically attached to a standard pen injector housing by a known attachment mechanism. The distal movement of a plunger or stopper within the medicament cartridge causes medication to be forced into a reservoir housing. The medicament cartridge is sealed by a septum and punctured by a septum penetrating needle cannula located within a reservoir or housing. Reservoir housing is preferably screwed onto the medicament cartridge although other attachment mechanism can be used. The pen needle delivery device can be a standard pen delivery device known in the industry so that the pen needle delivery device is not shown in detail. The pen needle assembly 18 as shown in FIG. 2 includes a needle hub 16 supporting a cannula 20, an outer cover 22, and an inner shield 24. A protective seal 26 is attached to the open end of the outer cover as shown in FIG. 2 to enclose the needle hub and cannula to maintain a clean and sterile condition. The seal 26 can be a label or other closure member that can be easily peeled from the outer cover to access the needle hub during use.

The pen needle delivery device 10 is connected to needle hub 16 shown in. FIG. 1 that has a connecting non-patient end with internal threads that screw onto a threaded end 14 of the delivery device 10. The needle cannula 20 extends from the patient end of the needle hub 16 for delivering the substance to the patient. The outer cover 22 can be provided to cover the needle cannula to protect the patient from accidental needle stick before and after use. The outer cover 22 includes ribs 23 to assist in gripping the outer cover during use. The inner shield 24 is provided over a post extending from the end of the needle hub 16 to enclose the cannula. During use, the needle hub 16 is connected to the pen delivery device and the inner shield is removed. After use, the outer cover is generally placed back on needle hub to cover the needle cannula. The needle hub with the cover is then removed from the pen needle delivery device and discarded. The term pen needle can refer to the needle hub and cannula.

The apparatus 30 is configured for receiving and supporting at least one pen needle assembly 18 in a position where the delivery device 10 can be coupled to the needle hub 16 for use for injecting the substance to the patient without the need for the user to handle the needle hub 16 with the exposed cannula. The used needle hub 16 and cannula 20 can then be positioned back into the outer cover 22 that is retained in the apparatus and separated from the delivery device. The used outer cover and needle hub and the inner shield are retained in the device.

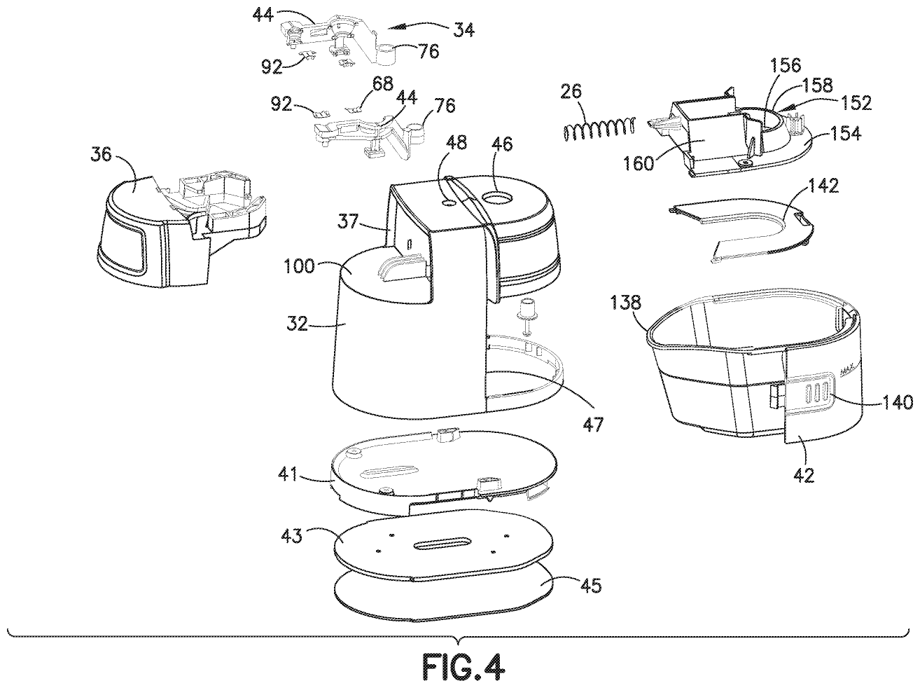

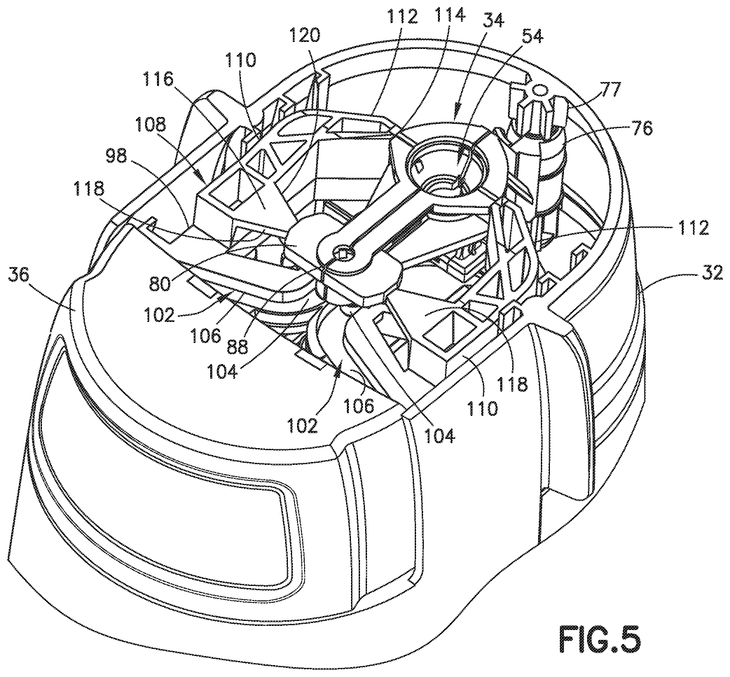

An apparatus 30 is configured for receiving a pen needle assembly 18 for coupling the pen needle hub 16 to the delivery pen and for separating the pen needle hub from the delivery pen after use. In the embodiment shown, the apparatus 30 includes a housing 32 with an inner cavity, a retaining and gripping mechanism 34 within the housing, and an actuator 36. The housing 32 as shown in FIG. 3 has a top end 38 and a bottom end 40. Bottom end 40 has a bottom wall 41 attached to the housing, a base plate 43, and with a non-slip pad 45 shown in FIG. 4. The nonslip pad can be molded directly onto the bottom plate or can be a nonslip material, such as an elastomeric material, applied to the bottom surface to retain the housing in a stable position during use on a table or other flat surface.

The housing 32 as shown in FIG. 4 has a sidewall with an opening 37 for the actuator 36. A support 152 for the gripping mechanism 34 is attached to the housing 32 for supporting the gripping mechanism and actuator 36. The support 152 has a bottom wall 154 attached to the housing above the tray 42. An opening 156 is formed in the bottom wall 154 to access the tray 42. A sidewall 158 extends from the bottom wall 154 at one end as shown in FIG. 4 for guiding the cover 22 into the tray 42. A side wall 160 extends from the bottom wall 154 for supporting the gripping mechanism 34 when sliding between the open position and closed position. In the embodiment shown, a tray 42 is provided to slide within an opening 47 in the housing and defining a receptacle or sharps container for the used pen needle assemblies.

Referring to FIGS. 4-6, the gripping mechanism 34 is provided for supporting and gripping a pen needle assembly for connecting the needle hub 16 to the delivery pen and disconnecting the needle hub from the delivery pen after the use. In the drawings, portions of the side wall and top wall of the housing 32 are not shown for clarity to illustrate the parts of the apparatus and gripping mechanism 34. The gripping mechanism 34 is a movable assembly that is actuated by the user to move the gripping mechanism between a first position for engaging and gripping the outer cover the pen needle assembly and a second position for releasing the outer cover.

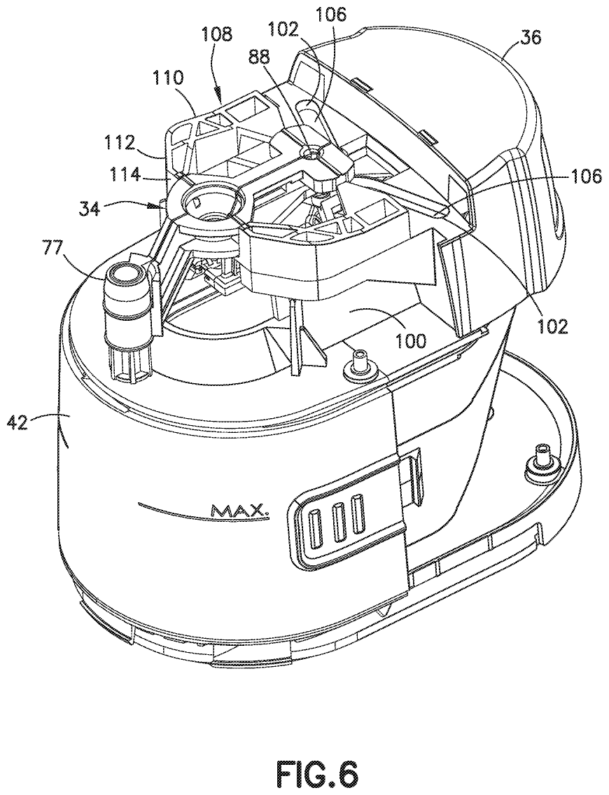

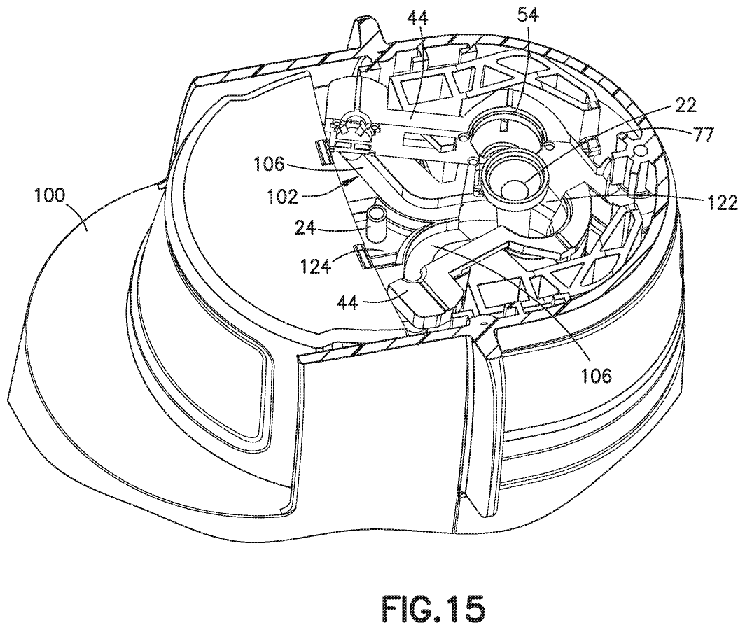

In the embodiment shown, the gripping mechanism 34 includes a first arm 44 and a second arm 44 that pivot between a first closed position shown in FIGS. 5 and 6 and a second open position shown FIG. 15. The arms 44 are mounted in the housing 32 and moved by the actuator 36. The housing 32 has an opening 46 with a dimension complementing the outer dimension of the pen needle assembly so that the pen needle assembly can slide through and into contact with the gripping mechanism 34. The housing has an opening 48 aligned with the gripping mechanism 34 for receiving the inner shield 24 of the pen needle.

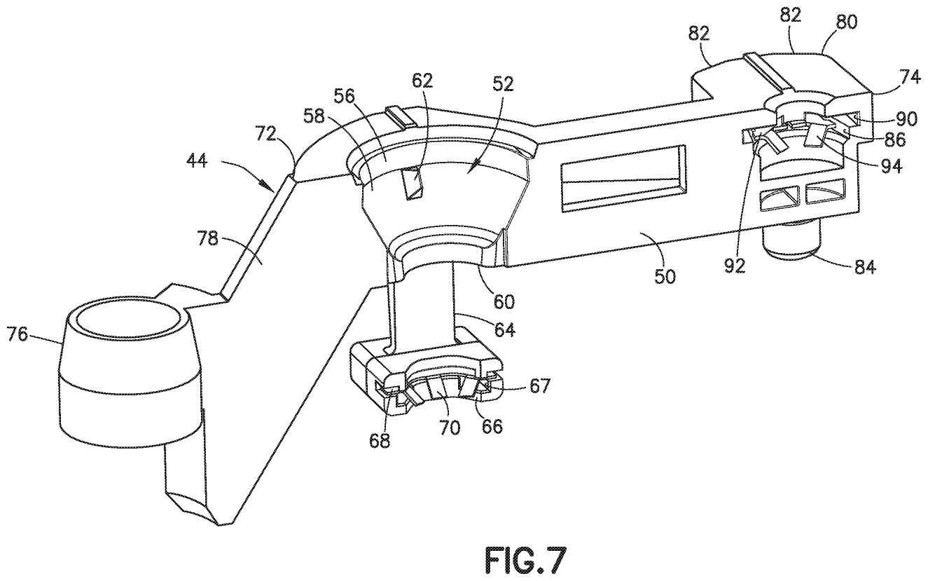

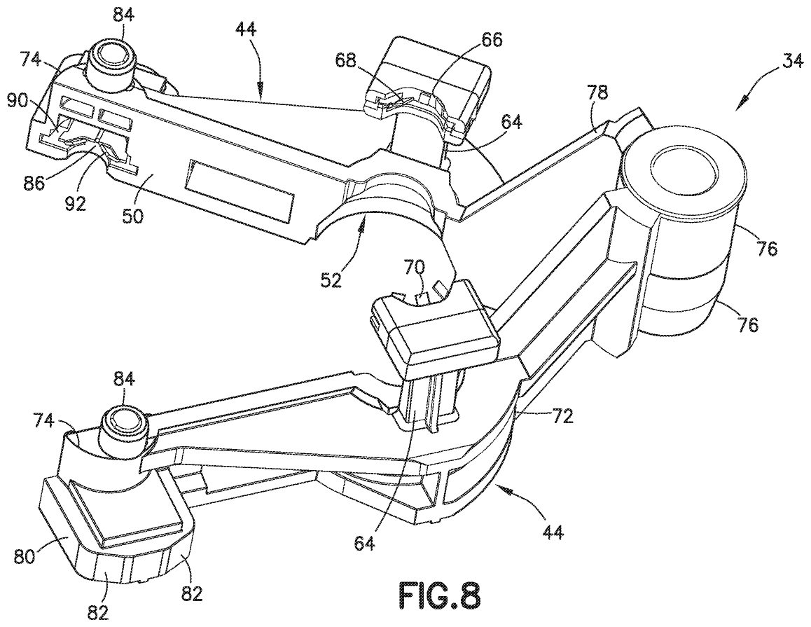

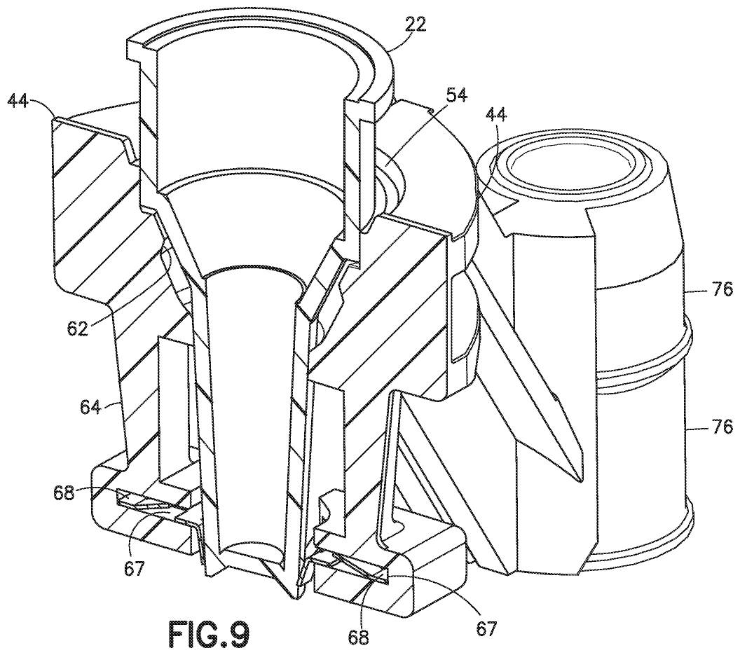

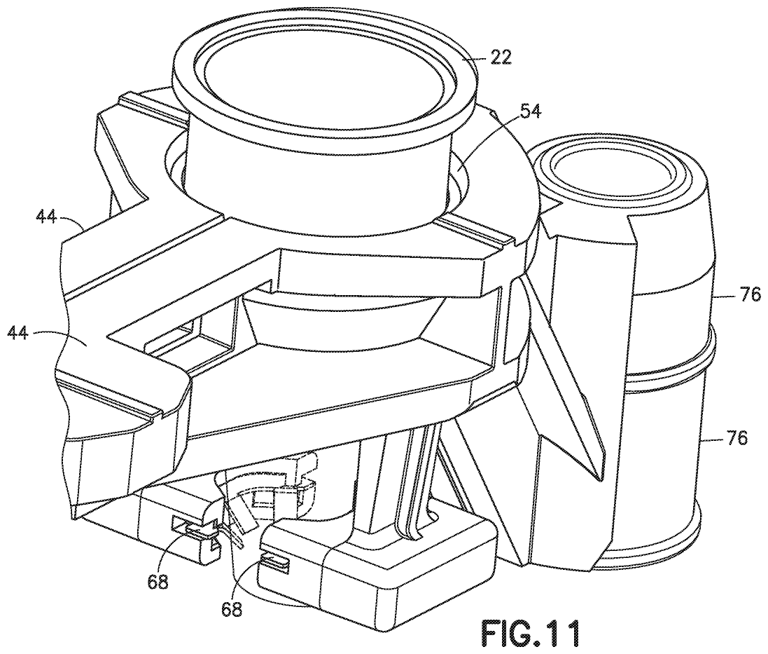

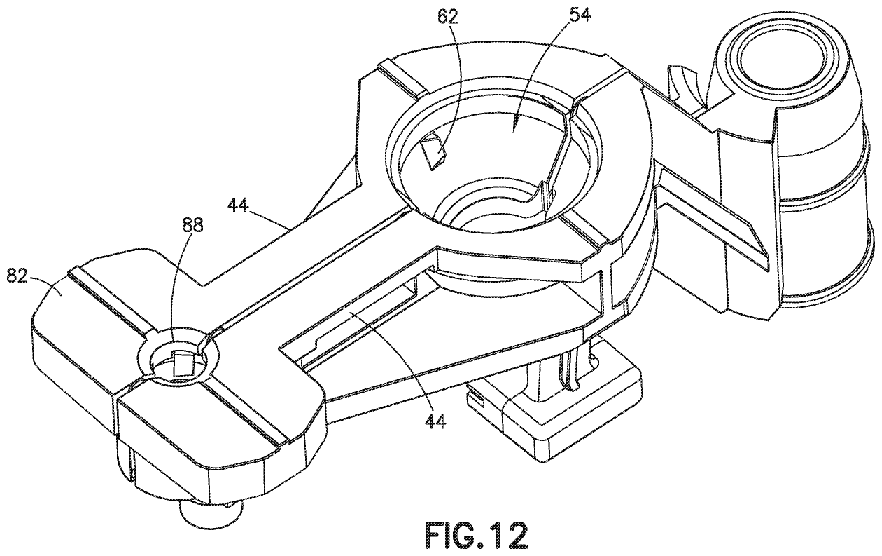

The arms 44 of the gripping mechanism 34 as shown FIG. 8 are substantially mirror images of each other and configured for pivoting between the closed position shown in FIG. 5 and the open position shown in FIG. 15. In the embodiment shown, the arms 44 are pivotally connected together and pivotally connected to the housing. Arms 44 have an inner mating surface 50 with a substantially semicircular open concave recess area 52 where the open concave recess area 52 of each arm 44 defines a substantially circular shaped well 54 for receiving a pen needle assembly. As shown in FIG. 7, the concave recess areas 52 have a substantially straight portion 56 extending in a longitudinal direction with respect to a longitudinal center axis of the well 54 shown in FIGS. 9 and 12 and forming a sidewall of the well 54. A bottom section of the well 54 is formed by an inclined section 58 forming a bottom opening 60 having a dimension less than the opening at the top end face of the gripping mechanism formed by arms 44. The opening 60 has a dimension to allow the narrow end portion of the outer cover to pass through. The inclined section 58 includes one or more projecting detents 62 to engage the ribs 23 on the outer cover of the pen needle assembly to resist rotation of the outer cover with respect to the gripping mechanism 34. The well 54 formed by the recess in the arms 44 has a shape and dimension complementing the shape and dimension of the outer cover of the pen needle assembly.

A leg 64 extends from a bottom side of each arm 44 that extends outwardly a distance to complement the longitudinal dimension of the outer cover of the pen needle assembly. The leg 64 has a distal end formed with a concave inner surface 66. The inner surface 66 includes a recess shown as a slot 67 with a gripping member 68 for gripping the outer surface of outer cover 22 of the pen needle assembly. In the embodiment shown, the gripping member 68 can be a spring clip including a plurality of teeth 70 encircling the open bottom end of arms 44. In the embodiment shown, the teeth 70 project at an inclined angle projecting toward the center axis of the opening 54 and extending away from the main body portion of the arms to resist pulling the outer cover from the well 54. In other embodiments, the gripping member can be integrally formed with the arms. The gripping member 68 is generally made of spring metal, plastic or other suitable material that is able to grip the outer surface of the cover during use. In the embodiment shown, the gripping member 68 is a metal spring clip having a tab to retain the member in the slot 67 and the teeth 70 having sharp edges to engage the outer cover and resist rotation until released by the gripping assembly.

The arms 44 have a first end 72 that are pivotally connected together as shown in FIG. 8 and a second free end 74. The first ends 72 have a hinge barrel 76 that are pivotally connected to a pivot boss 77 shown as a hinge pin fixed to the housing as shown in FIG. 5 to define a fixed pivot axis. In the embodiment shown, an inclined portion 78 extends between the arms 44 and the hinge barrel 76 to space the body of the arms from the upper surface of the housing to provide a space for the sliding movement of the actuator 36. The arms 44 are pivotally mounted to the housing to pivot relative to each other between a closed position shown in FIG. 5 and an open position shown in FIG. 15.

The free ends 74 of the arms 44 have an outwardly extending member 80 with inclined surfaces 82. A bottom face of the arms each include a cam follower 84 projecting in a generally axial direction parallel to the pivot axis of the hinge barrels 76 and parallel to the axis of the well 54. As shown in FIGS. 8, the cam followers 84 have a substantially cylindrical shape. The cam follower 84 can be a rotating bearing attached to the arm by a screw or other fastener. In other embodiments, the cam follower can be a pin or rod that slides within the cam surface of the actuator.

The free end 74 of each arm 44 has a semicircular concave recess 86 that form a well 88 between the two arms 44 when the arms are in the closed position. The well 88 has a dimension for receiving the inner shield of the pen needle. A slot 90 is formed on the inner surface of the recess 86 for receiving a spring member 92 shown in FIG. 13 having inwardly extending teeth 94 that are angled in a downward direction with respect the arms and the housing to grip the inner shield.

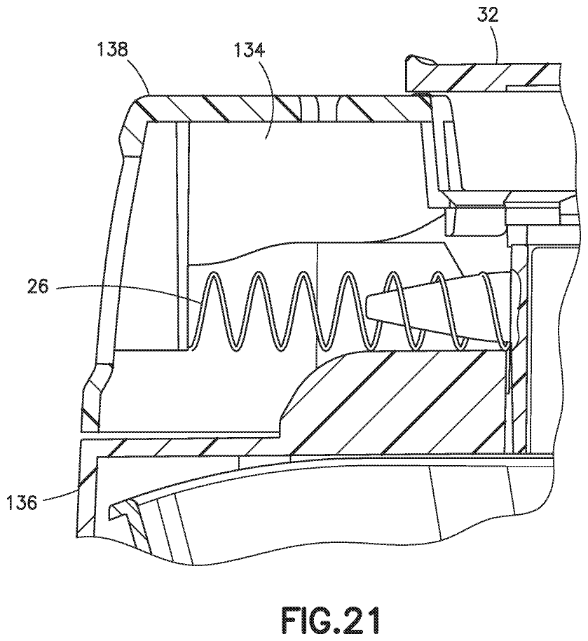

The actuator 36 in the embodiment shown is coupled to the housing 32 for sliding on the top wall 100 with respect to the housing in a substantially linear direction perpendicular to the pivot axis of the arms 44 and perpendicular to the axis of the pen needle when received in the well. As shown in FIGS. 5 and 6, the actuator 36 slides in an opening 37 in the side of the housing as shown in FIG. 4. The actuator 36 has an end wall 96 facing outwardly from the housing and a body 98 positioned within the housing. As shown in FIG. 21, the actuator 36 is spring biased outwardly with respect to the housing by a spring 126.

The body 98 has a substantially planar shape for sliding on the wall 160 of the support 152 and below the arms 44 as shown in FIGS. 4, 5 and 15. The body 98 has a top surface formed with cam surfaces for actuating the arms 44. In the embodiment shown, the top surface of the body 98 includes grooves 102 forming the cam surfaces that cooperate with the cam follower 84 of the respective arms 44. As shown in FIGS. 5 and 6, the cam grooves 102 have a first section 104 extending in a linear direction with respect to the direction of the sliding movement and in direction extending toward the arms 44. A second section 106 extends outwardly from the respective first section 102 and terminates toward the respective outer side edges of the body 98. The first sections 104 of the respective grooves 102 are substantially parallel to each other as shown in FIG. 5. The second sections 106 diverge outwardly from the respective first section 104 in opposite directions and at an inclined angle with respect to the first section 104 and the direction of the sliding movement of the actuator 36.

As shown in FIG. 5, the body 98 has upwardly extending walls 108 positioned on the opposite sides edges. The walls 108 have a main portion 110 extending in a longitudinal direction with respect to the body 98 and are spaced outwardly from the arms 44 of the gipping mechanism. The main portion 110 has an outer face for sliding along the inner surface of the sidewall of the body and an inner surface facing the arms 44. A first end of the main portion 110 has a leg 112 extending inwardly at an inclined angle relative to the longitudinal dimension of the main portion 110. An end surface 114 of the leg 112 is positioned to contact an outer surface of the first end of the respective arm 44 when the actuator 36 is in the retracted position and the arms 44 are closed as shown in FIG. 5. A second end of the wall 108 has an inwardly extending tab 116 having an inclined leading surface 118 and an inclined trailing surface 120 and an end face between the inclined surfaces 118 and 120. The tab 116 has a dimension where the end face contacts the outer surface of the member 80 on the respective arm 44 when the arms 44 are in the closed position shown in FIG. 5.

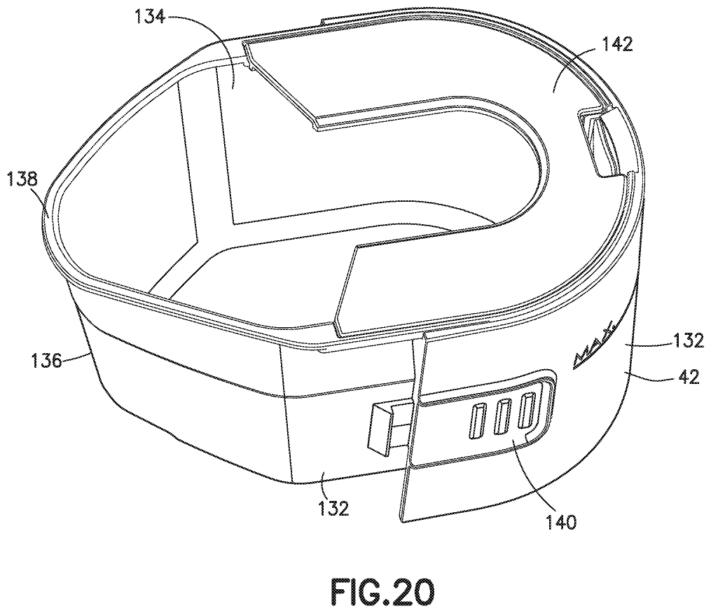

The tray 42 as shown in FIG. 20 has a side wall 130, a bottom wall 132 and an open top end 134. The tray 42 has an end portion 136 with a dimension for fitting within the housing below the gripping mechanism for receiving the used pen needles when released by the gripping mechanism. As shown the end portion forms a spout 138 for easy dispensing of the used pen needles. Clips 140 are provide on each side for coupling the tray to the housing during use. A lip 142 is attached to the open end of the tray 42 as shown in. FIG. 4.

The body 98 is configured for actuating the arms 44 so that the needle hub and inner shield captured between the arms fall into the tray 42 for disposal. The body 98 has open area 122 that aligns with the well 54 and an open area 124 that aligns with the well 88 when the arms 44 are in the open position so that the pen needle and inner shield drop into the tray as shown in FIG. 15.

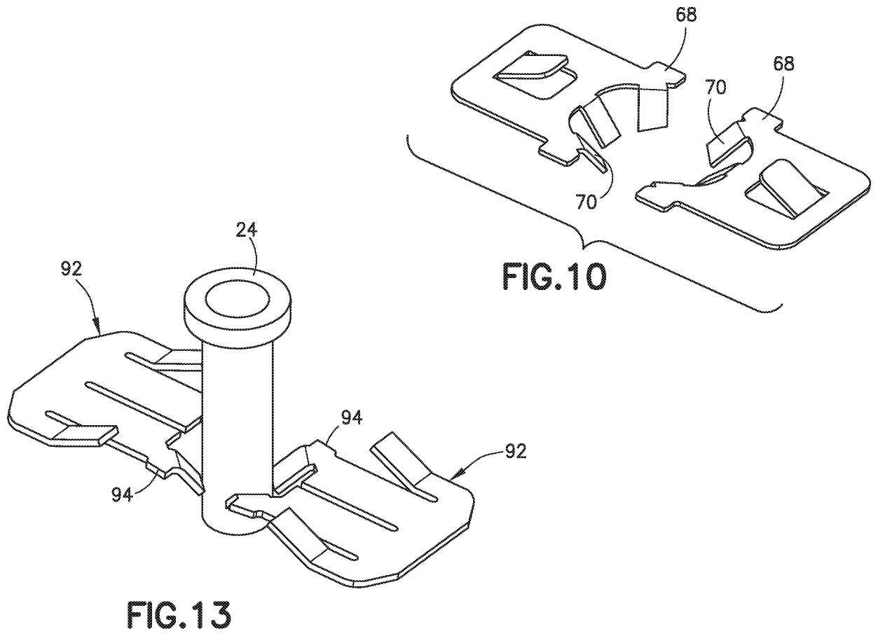

The assembly is able to receive a pen needle assembly to assist the user in attaching the needle hub to the delivery pen without the need for the user to handle the needle hub and exposed cannula. The pen needle assembly is placed into the opening 46 in the housing 32 so that the pen needle assembly passes downwardly into the well 54 defined by the recesses in the arms 44 as shown FIG. 9 and FIG. 10. The label is removed from the outer cover before or after placing the pen needle assembly in the well. The pen needle assembly is retained by the arms 44 and the teeth 70 engaging the outer surface at the bottom outer cover 22 as shown in. FIG. 9. The detent 62 on the inclined surface of the respective arms 44 engages the ribs 23 on the outer surface of the outer cover to resist rotation of the outer cover relative to the arms.

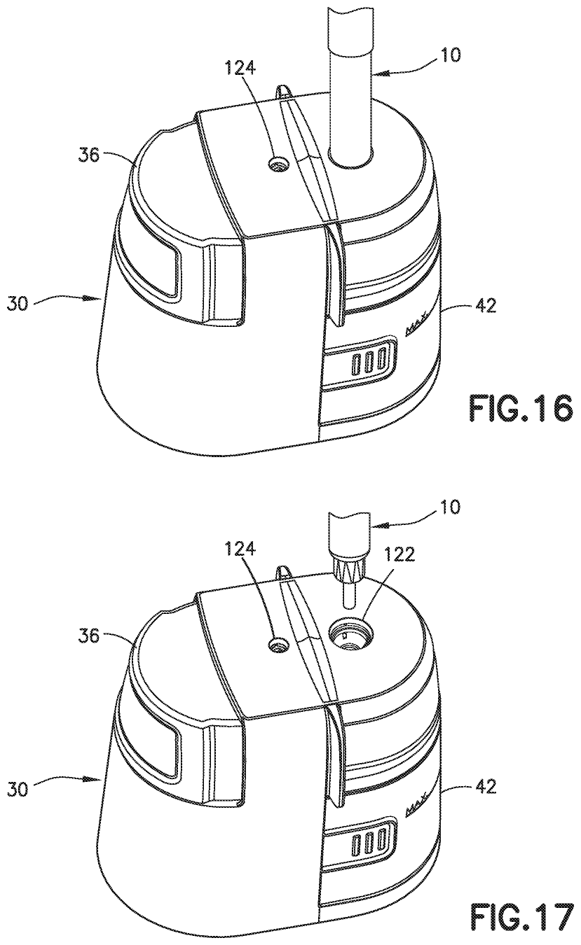

As shown in FIG. 16, a delivery pen is then inserted through the opening in the housing to engage the needle hub of the pen needle assembly. The delivery pen is rotated to engage the internal threads on the needle hub to couple to the delivery pen. As shown in FIG. 17, the delivery pen with the attached needle hub 16 can be retracted from the outer cover 22 leaving the outer cover retained in the well 54 between the arms 44 in the housing.

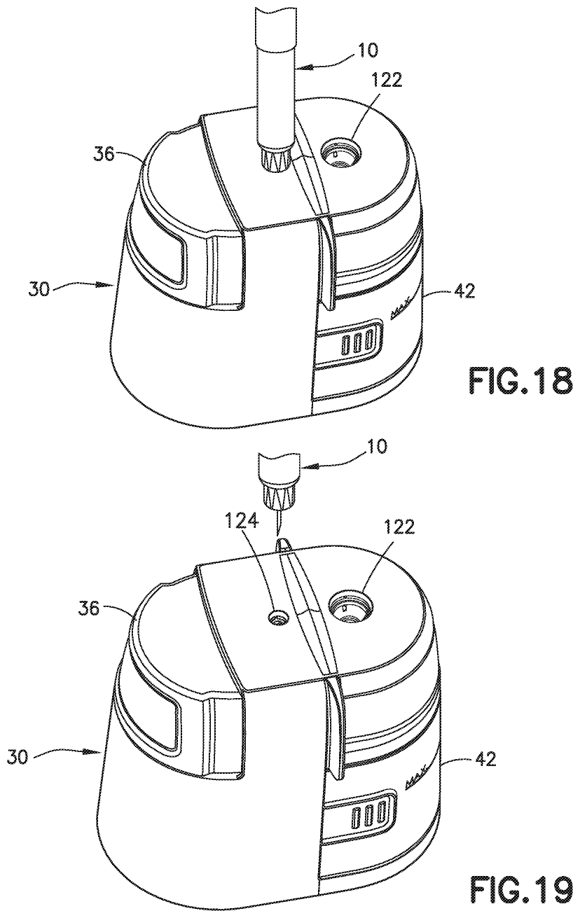

The inner shield 24 is attached to the needle hub 16 to cover the cannula 20 when separated from the outer cover. The delivery pen is then inserted into the opening 48 as shown in. FIG. 18 where inner shield 24 engages the teeth in the well 88 to grip the inner shield and resist upward movement. The delivery pen can then be extracted leaving the inner shield 24 retained in well 88 between of the arms 44.

After use of the delivery pen and the pen needle assembly 18, the delivery pen can then be inserted through the opening in the housing where the needle hub 16 is again positioned in outer cover 22 that is retained in the well 54 between the arms 44. The delivery pen is rotated to disconnect the delivery pen from the internal threads of the needle hub and removed from the apparatus.

As shown in FIG. 5, the tabs 116 of the wall 108 engage the members 80 at the ends of the arms 44 and the end of the second section 112 engage the outer surface of the arms 44 around the well 54 to assist in retaining the arms in the closed position. The actuator 36 is then pushed to slide the body 98 in a linear direction with respect to the arms 44. The sliding movement of the body 98 causes the cam surfaces of the grooves 102 that engage the respective earn follower 84 on the arms 44 to pivot the arms 44 outwardly to the position shown in FIG. 15 to release the outer cover 22 that contains the used needle hub 16 and the inner shield 24. During the sliding movement of the body of the actuator 36, the tabs 116 and the inner ends of the legs 112 of the body 98 slide forward relative to the arms 44. As shown, the leg 112 slides past the first end of the arms and the tab 116 slides past the member 80 to allow the arms to pivot outwardly by the cam grooves 102 and the can follower 84.

The used outer cover 22 with the needle hub 16 are released from the arms 44 to pass through the opening into the tray 42 positioned in the housing below the arms 44 where the used pen needles are collected for disposal. The actuator is returned to the original position by the spring 26 or other biasing member. The cam surfaces of the grooves 102 return the arms to the closed position for reuse with a new pen needle. The end surfaces 114 of the legs 12 and the inclined surface 82 of the member 80 of actuator 98 contact the respective arms 44 to assist in moving the arms to the closed position. The steps can be repeated by placing a new pen needle assembly in the housing and the delivery pen can be attached to the needle hub.

In other embodiments, it may be desirable to place the inner shield back onto the needle hub 16 for later use after the delivery device has been prepared for use. The inner shield 24 can be placed back onto the post of the needle hub 16 by inserting the delivery pen and needle hub back into the apparatus as shown. The delivery pen with the needle hub and inner shield 24 attached can be withdrawn and stored for later use.

In a further embodiment, the outer cover 22 can be re-attached to the hub 16 without separating the needle hub from the delivery pen. The delivery pen with the attached needle hub 16 can be inserted through the opening and the housing so that the needle hub 16 is received in the outer cover 22. The actuator 36 can then be pushed to the actuated position shown in FIG. 15 to separate the arms 44 to release the outer cover 22. The delivery pen can then be withdrawn with the outer cover protecting the cannula to avoid inadvertent needle stick.

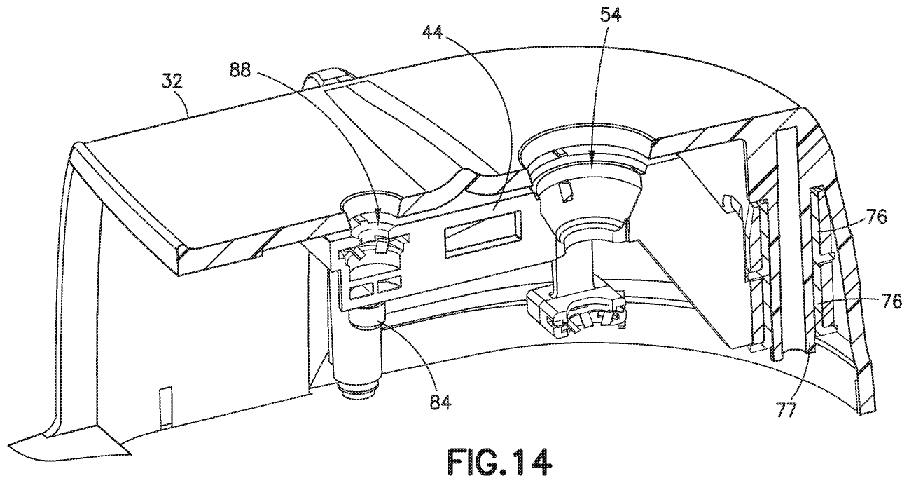

In the embodiment of FIGS. 3-20, the apparatus is shown with a single opening and well for receiving a single pen needle assembly. In alternative embodiments, the assembly can have more than one opening and opposing arms for accommodating a plurality of pen needle assemblies. In another embodiment the assembly can include a housing with a plurality of wells for receiving a respective pen needle assembly. An actuator button can be provided for each respective well for operating a gripping mechanism for retaining the outer cover of the pen needle assembly.

The foregoing embodiments and advantages are merely exemplary and are not to be construed as limiting the scope of the present invention. The description of an exemplary embodiment of the present invention is intended to be illustrative, and not to limit the scope of the present invention.

Various modifications, alternatives, and variations will be apparent to those of ordinary skill in the art, and are intended to fall within the scope of the invention. It is particularly noted that the features of different embodiments and claims may be combined with each other as long as they do not contradict each other. Accordingly, all such modifications are intended to be included within the scope of this invention as defined in the appended claims and their equivalents.

1. A pen needle apparatus comprising:
a housing having an inner cavity, a top end, and a bottom end;
a gripping mechanism having a first pivoting arm configured for gripping an outer cover of a pen needle assembly when the first pivoting arm is in a first closed position and releasing the outer cover when the first pivoting arm is in a second open position; and
an actuator connected to said first pivoting arm to pivot said first pivoting arm from said first position to said second position and release the outer cover whereby the outer cover passes into said inner cavity.

2. The pen needle apparatus of claim 1, wherein said gripping mechanism is configured for receiving and gripping an inner shield of the pen needle.

3. The pen needle apparatus of claim. 2, wherein said first pivoting arm in said second position releases the inner shield and the outer cover.

4. The pen needle apparatus of claim 1, wherein said gripping mechanism further comprises a second pivoting arm, each said first pivoting arm and second pivoting arm being coupled to said actuator, and where movement of said actuator pivots said first pivoting arm and second pivoting arm between said first position to grip the outer cover and said second position to release the outer cover.

5. The pen needle apparatus of claim 4, wherein said actuator includes a first cam surface engaging said first pivoting arm and a second cam surface engaging said second pivoting arm.

6. The pen needle apparatus of claim 5, wherein said first pivoting arm and second pivoting arm are pivotally connected together and pivotally connected to said housing.

7. The pen needle apparatus of claim 5, wherein said first pivoting arm and second pivoting arm have a first recess aligned with each other to form a first well when said first pivoting arm and second pivoting arm are in said first closed position and where said first well has a dimension for receiving said outer cover.

8. The pen needle apparatus of claim 7, wherein said first arm and second arm have a second recess aligned with each other for forming a second well when said arms are in the first closed position, and where said second well has a dimension for receiving an inner shield of the pen needle assembly.

9. The pen needle apparatus of claim 5, wherein said first cam surface has a first section extending in a longitudinal direction with respect to a direction of movement of said actuator, and a second section extending at an inclined angle with respect to said first section whereby linear movement of said actuator pivots said first pivoting arm, and wherein said second cam surface has a first section extending in a longitudinal direction with respect to a direction of movement of said actuator, and a second section extending at an inclined angle with respect to said first section whereby linear movement of said actuator pivots said second pivoting arm. away from said first pivoting arm.

10. The pen needle apparatus of claim 5, wherein said actuator is coupled to said housing for linear movement from a first position where said first pivoting arm and second pivoting arm are in the first position, and a second position where said first pivoting arm and second pivoting arm are in said second open position.

11. The pen needle apparatus of claim 5, wherein said first pivoting arm and second pivoting arm have a first end pivotally coupled to said housing, and a second free end connected to said actuator to pivot said second free ends away from each other.

12. A pen needle apparatus for storing a used pen needle assembly, comprising:
a housing having an inner cavity, a bottom end, and a top end with an opening for receiving a pen needle assembly;
a gripping mechanism having a first pivoting arm pivotally connected to said housing and a second pivoting arm pivotally connected to said housing, said first pivoting arm and second pivoting arm configured for gripping an outer cover of the pen needle assembly when said first pivoting arm and second pivoting arm are in a first closed position and pivoting to a second open position to release the outer cover; and
an actuator coupled to said housing for sliding in a transverse direction relative to said housing, said actuator coupled to said first pivoting arm and second pivoting arm for pivoting said first pivoting arm and second pivoting arm from said first closed position to said second open position to release the outer cover from said apparatus.

13. The pen needle apparatus of claim 12, wherein said first pivoting arm is pivotally coupled to said a second pivoting arm.

14. The pen needle apparatus of claim 12, wherein said actuator includes a first cam surface engaging said first pivoting arm and a second cam surface engaging said second pivoting arm.

15. The pen needle apparatus of claim 14, wherein said first pivoting arm and second pivoting arm have a first end pivotally coupled together, and a second free end.

16. The pen needle apparatus of claim 15, wherein said second free end of said first pivoting arm and second pivoting arm are operatively connected to said actuator.

17. The pen needle apparatus of claim 15, wherein said first pivoting arm and second pivoting arm are pivotally connected together and pivotally connected to said housing by a fixed pivot boss.

18. The pen needle apparatus of claim 12, wherein said first pivoting arm and second pivoting arm each have a first recess aligned with each other to form a first well when said first pivoting arm and second pivoting arm are in said first closed position and where said first well has a dimension for receiving said outer cover.

19. The pen needle apparatus of claim 18, wherein said first pivoting arm and second pivoting arm have a second recess aligned with each other for forming a second well when said first pivoting arm and second pivoting arm are in the first closed position, and where said second well has a dimension for receiving an inner shield of the pen needle assembly.

20. The pen needle apparatus of claim 15, wherein said first cam surface has a first section extending in a longitudinal direction with respect to a direction of movement of said actuator, and a second section extending at an inclined angle with respect to said first section whereby linear movement of said actuator pivots said first pivoting arm, and wherein said second cam surface has a first section extending in a longitudinal direction with respect to a direction of movement of said actuator, and a second section extending at an inclined angle with respect to said first section whereby linear movement of said actuator pivots said second pivoting arm away from said first pivoting arm.

21. A method of coupling a pen needle to a delivery pen, said method comprising
positioning a pen needle assembly in a first well of a pen needle apparatus formed between a first pivoting arm and a second pivoting arm of a gripping mechanism for gripping an outer cover of the pen needle assembly, and attaching the delivery pen to the pen needle assembly;
removing the pen and a needle hub from an outer cover and inserting an inner shield of the pen needle into a second well in the apparatus formed between the first pivoting arm and the second pivoting arm to grip the outer shield, and removing the inner shield from the needle hub of the pen needle assembly;
inserting the needle hub after use into the outer cover and separating the pen from the needle hub; and.
depressing an actuator to release the outer cover and needle hub from the first and second pivoting arms.

22. The method of claim 21, wherein said cam member of said actuator includes a first cam surface engaging said first pivoting arm, and a second cam surface engaging said second pivoting arm, and where movement of said actuator moves said first pivoting arm and second pivoting arm away from each other to release the outer cover of the pen needle assembly.

23. The method of claim 21, wherein said first pivoting arm and second pivoting arm have a first recess forming said first well when said first pivoting arm and second pivoting arm are in a first closed position, and a second recess forming said second well when said pivoting arms are in the first closed position.

24. The method of claim 21, wherein said actuator includes a first cam surface operatively connected to said first pivoting arm, and a second cam surface operatively connected to said second pivoting arm.
